(19) United States
(12) Patent Application Publication
(10) Pub. No.: US 2008/0065175 A1
(43) Pub. Date: Mar. 13, 2008

(54) Therapeutic radiation device

(76) Inventors: Russell J. Redmond, Goleta, CA (US); Claude Vidal, Santa Barbara, CA (US)

Correspondence Address: JAMES E. BRUNTON, ESQ., P. O. BOX 29000, GLENDALE, CA, 91209, US

(21) Appl. No.: 11/517,963

(22) Filed: Sep. 9, 2006

(51) Int. Cl. A61N 5/00

(52) U.S. Cl. 607/86

(57) ABSTRACT

It is an object of the present invention to provide a hand-held radiation device for treating bacterial, viral, fungal and parasitic infections found on the skin of a patient and in various of the body's anatomical orifices. The device of the invention is particularly effective in treating Methicillin resistant staphlococcus aureus (MRSA) colonies in the nose and on the skin surface of a patient. The device includes a reusable UV light source and a UV-transparent disposable cover for covering the probe portion of the reusable UV light source. The device further includes a combination probe cover ejector and disabling assembly for safely ejecting the probe cover after use without the necessity of the operator touching the contaminated probe cover and for disabling the device if the cover is not in place over the probe.

### BACKGROUND OF THE INVENTION

1. Field of the Invention

The present invention relates generally to irradiation devices for therapeutic purposes. More particularly, the invention concerns a readily portable, hand-held irradiation treatment device for treating infections that occur on the skin of the patient and in various of the body's anatomical orifices.

2. Discussion of the Prior Art

Ultraviolet (UV) light has long been used for disinfection and sterilization. In recent years, the widespread availability of low to medium pressure mercury bulbs has led to the development of devices which use UV-C for air purification and to decontaminate water supplies. UV-C has also found some limited use in food processing and in medical device sterilization. UV-C is a high frequency wavelength of light within the ultraviolet band and has been shown to be the most bactericidal type of ultraviolet light.

By way of background, UV light consists of high energy photons, which occupy the 200 to 400 nanometer wavelengths of the electromagnetic spectrum. This means that UV light emits slightly less energy than soft X-ray radiation, but significantly more than visible light. UV energy does not directly kill pathogens, but rather causes a photochemical reaction within the genetic structure which inhibits the ability of the pathogens to reproduce, therefore, in effect, killing the pathogen.

The amount of energy delivered by UV light is inversely proportional to its wavelength; therefore, the shorter the wavelength, the greater the energy produced. In general, the UV light portion of the spectrum is made up of three segments; UV-A (315-400 nm), used for sun-tanning lamps, UV-B (280-315 nm) and UV-C (200-280 nm). The UV-B and, as previously mentioned, UV-C regions contain wavelengths with the best germicidal action. Studies have shown that the wavelengths most effective in killing microbes are between 250-265 nm. This value corresponds nicely with the light energy output of a typical, commercially available UV-C germicidal lamp, which produces most of its energy output in the range of 254 nm.

An application of particular interest for devices of the present invention is the treatment of Staphylococcus aureus. Staphylococcus aureus (SA) is a bacteria commonly found on the skin and in the nose of healthy people. Occasionally, SA can enter the body and cause various types of infections. These infections can be minor, i.e., pimples or boils, or more serious and, in some cases, fatal, i.e., in blood infections or pneumonia. SA is a common organism and can be found in the nostrils of up to 30% of the population.

Methicillin resistant staphylococcus aureus (MRSA) are staphylococci that are resistant to the antibiotic methicillin and other commonly used antibiotics such as penicillin and cephalosporins. These germs have a unique gene that causes them to be unaffected by all but the highest concentrations of antibiotics. Over the past 30 years, the overuse of antibiotics has resulted in an increase in the incidence of MRSA in hospitals and, now, in the community at large. Studies indicate that since 1968, when the first reported MRSA cases were reported in hospitals, the proportion of MRSA causing infection in hospitalized patients has risen from 2% in 1974 to about 40% in 1997, and it is still rising! Over the past twenty years, infections with MRSA have been limited primarily to patients in hospitals or long-term care facilities. However, recent reports of community-acquired MRSA infections raise concern that the problem is now spreading to a larger population.

As with most SA, MRSA bacteria reside on the skin surface and in the nose. Colonies within the nose are usually within 1 cm of the nostril opening. If these colonies are transferred from the nose to an open cut, a MRSA skin infection may result and, if they are transferred into the blood stream via a more serious cut or skin break; then septicemia may result.

Published literature also indicates that the germicidal properties of short-wave UV light are also effective in killing viruses of the character that first congregate in and then multiply in the mucous membranes of the nose before they spread to other parts of the body. With this in mind, use of the devices of the present invention in the manner presently to be described tend to eliminate or at least reduce the severity of certain viral infections, such as avian influenza.

As previously mentioned, in addition to having clinical application in treating infections in the nostril and on the surface of the patient's skin, the present invention is also effective in treating bacterial, viral, fungal and parasitic infections found in various of the body's anatomical orifices. By way of non-limiting examples, applications of interest for devices of the present invention include the treatment of strep throat in the mouth of the patient; for treating various sexually transmitted diseases, such as Gonococcal and Non-gonococcal Urethritis and various other urinary tract infections in the urethra; for treating common yeast infections (fungal) in the vagina; for treating various sexually transmitted diseases, such as Gonococcal and Non-gonococcal Urethritis and parasitic infections in the rectum and for treating infections in the external ear canal of the patient.

The probe covers are designed so that they slightly dilate the lumen of the orifice into which they are inserted. This dilation sets the distance from light source to tissue surface very accurately as the dilated tissue conforms to the probe cover, giving uniform coverage of the light energy to the tissue.

Pre-setting the distance between light source and tissue, which would require the probe cover to touch the infected skin, is quite different than simply holding a UV light over the tissue as this method does not stretch the tissue. Stretching the tissue so that the entire surface is exposed to the light energy is critical to the successful germicidal use of UV light as it is a ‘line of sight’ process. Creases or folds can hide bacteria from exposure to the light energy, preventing them from receiving a lethal dose of irradiation.

### SUMMARY OF THE INVENTION

It is an object of the present invention to provide a novel hand-held UV radiation device that is particularly effective in treating bacterial, viral, fungal and parasitic infections found in various of the body's anatomical orifices.

It is another object of the present invention to provide a device of the aforementioned character that is particularly effective in treating MRSA colonies in the nose and on the skin surface of a patient.

Another object of the invention is to provide a device of the character described in the preceding paragraph that can eliminate, or greatly reduce, the MRSA colonies without significantly damaging the underlying tissue of the patient.

Another object of the present invention is to provide a novel hand-held radiation device that is effective in eliminating, or at least significantly reducing, the severity of certain viral infections such as avian influenza.

Another object of the invention is to provide a device as described in the preceding paragraphs that includes a reusable UV light source and a UV-transparent disposable cover for covering the probe portion of the reusable UV light source.

Another object of the invention is to provide a device as described in the preceding paragraph in which the disposable probe cover is uniquely designed to slightly dilate the lumen of the orifice into which it is inserted. This dilation sets the distance from light source to tissue surface very accurately as the dilated tissue conforms to the probe cover, giving uniform coverage of the light energy to the tissue.

Another object of the invention is to provide a device of the type described in which the disposable probe cover is uniquely designed to shield the tissue surrounding the radiation site so that only the radiation site receives the germicidal UV energy.

Another object of the invention is to provide a device as described in the preceding paragraphs that is effective in the treatment of strep throat in the mouth of the patient.

Another object of the invention is to provide a device as described in the preceding paragraphs for treating various sexually transmitted diseases, such as Gonococcal and Non-gonococcal Urethritis and various other urinary tract infections in the urethra.

Another object of the invention is to provide a device as described in the preceding paragraphs for treating for treating common yeast infections (fungal) in the vagina.

Another object of the invention is to provide a device as described in the preceding paragraphs for treating various sexually transmitted diseases, such as Gonococcal and Non-gonococcal Urethritis and parasitic infections in the rectum.

Another object of the invention is to provide a device as described in the preceding paragraphs for treating infections in the external ear canal of the patient.

Another object of the invention is to provide a hand-held radiation device of the character described that includes a safety, disabling assembly that is so constructed and arranged to permit the UV radiation source to be energized only when the probe cover is in position over the device probe.

Another object of the invention is to provide a device of the type described in the preceding paragraphs that includes a novel, easy-to-use ejection mechanism for safely ejecting the probe cover after use without the necessity of the operator touching the contaminated probe cover.

Another object of the invention is to provide a device of the character described that is compact, light weight and of simple construction, is easy-to-use and can be inexpensively manufactured.

### BRIEF DESCRIPTION OF THE DRAWINGS

FIG. 1 is a generally perspective view of one form of the irradiation treatment device of the present invention.

FIG. 2 is an enlarged, fragmentary, perspective view of the area designated in FIG. 1 as “2”.

FIG. 3 is an enlarged, generally perspective view of one form of a disposable probe cover usable with the apparatus shown in FIG. 1 of the drawings.

FIG. 4 is a generally perspective, foreshortened, exploded view of the device illustrated in FIG. 1, here shown interconnected with an energy source and depicted with the disposable probe cover in position over the forward portion of the device.

FIG. 5 is a generally schematic view illustrating the interconnection of the various operating components of the device shown in FIG. 1.

FIG. 6 is a generally schematic view illustrating the interconnection of the various operating components of an alternate form of treatment device of the invention.

FIG. 7 is a side-elevational view of one form of the disposable probe cover of the apparatus of the present invention.

FIG. 8 is a cross-sectional view taken along lines 8-8 and FIG. 7.

FIG. 9 is a side-elevational view of an alternate form of the disposable probe cover of the apparatus of the invention.

FIG. 10 is a side-elevational view of another alternate form of the disposable probe cover of the apparatus of the invention.

FIG. 11 is a side-elevational view of still another form of the disposable probe cover of the apparatus of the invention.

FIG. 12 is a top plan view illustrating the device of the invention being used with still another form of the disposable probe cover designed to treat the skin of the patient.

FIG. 13 is a generally illustrative view showing the device of the invention in position preparatory to treating bacterial colonies in the patient's nose.

FIG. 14 is a generally illustrative view showing the device of the invention moved into position to treat bacterial colonies in the patient's nose.

### DESCRIPTION OF THE INVENTION

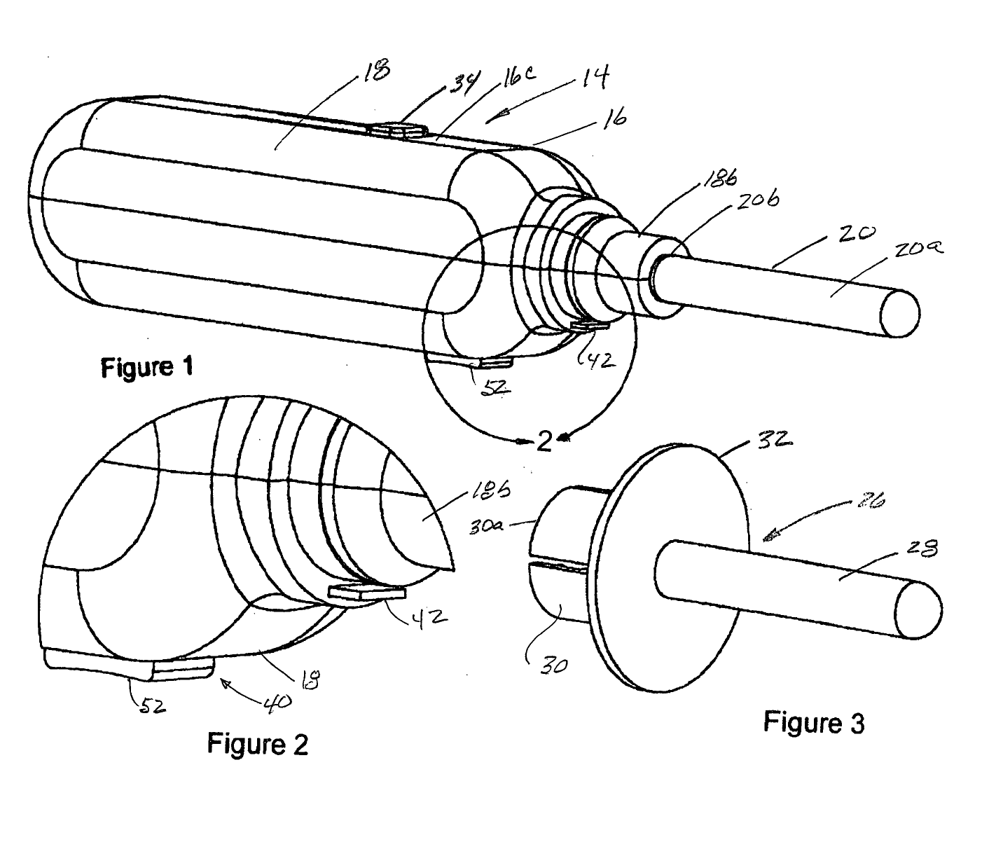

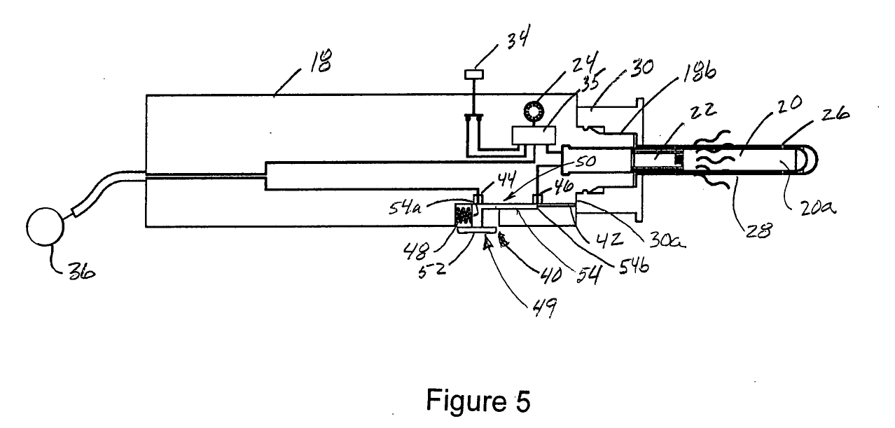

Referring to the drawings and particularly to FIGS. 1 through 5, one form of the novel irradiation device of the invention for treating bacterial colonies on the skin of the patient and in various body orifices, such as the mouth, the urethra, the vagina, the rectum and the external ear canal is there shown and generally designated by the numeral 14. This form of the device here comprises a housing 16 having a hollow, generally cylindrical-shaped body portion 18 and an elongated probe portion 20 that is connected to housing portion 18 and extends outwardly therefrom. Probe portion 20, which has a forward irradiating portion 20a and rearward portion 20b, is connected to the forward hub portion 18b of body portion 18 in the manner shown in FIGS. 1 and 5. Operably associated with probe 20 is a conventional radiation source 22 for emitting UV radiation in a direction toward the elongated probe portion 20a. Disposed within housing 16 is a timer 24, the function of which will presently be described (FIG. 5).

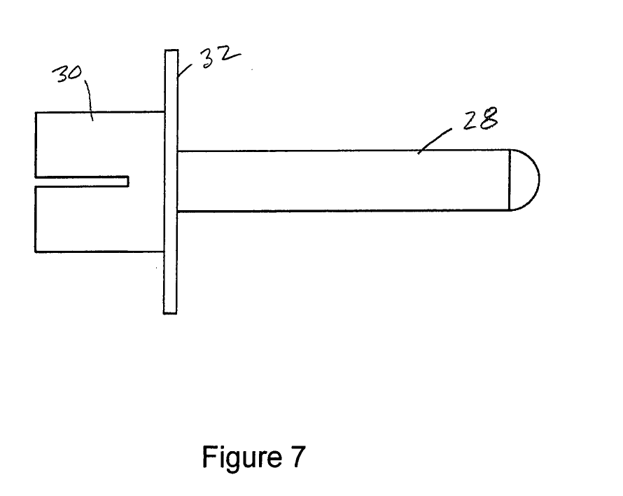

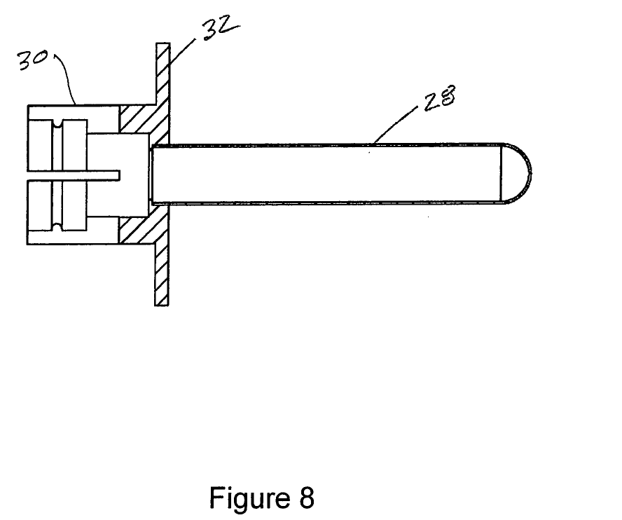

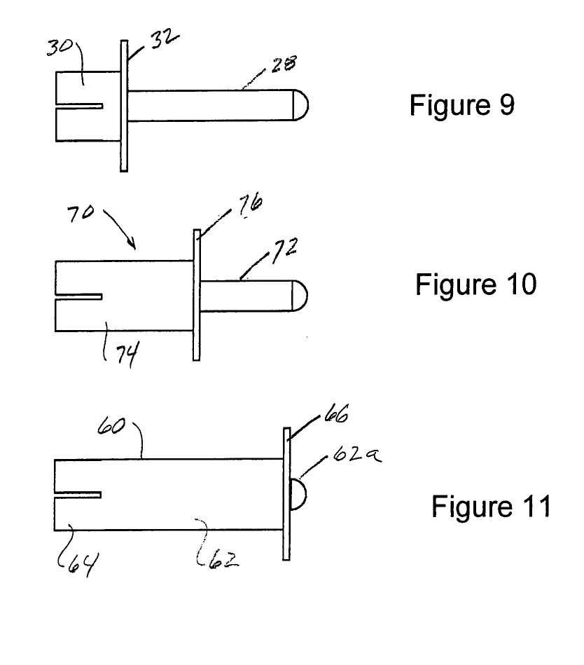

Removably connected to the forward irradiating portion of the elongated probe is a uniquely constructed, substantially UV radiation-transparent probe cover 26. As best seen in FIGS. 3, 7 and 8, probe cover 26 includes a generally tubular-shaped forward portion 28 that covers the forward portion of the probe and a generally tubular-shaped, slotted connector portion 30 that is telescopically receivable over the forward hub portion 18b of housing portion 18. Forward portion 28 can be constructed from polypropylene, certain varieties of Teflon that are transparent to UV light in the UV-C range (200-280 nm) and other amorphous fluropolymers. Disposed intermediate forward portion 28 and connector portion 30 is an enlarged diameter radiation shield 32 that functions to shield the user's hand from radiation (see also FIGS. 8 and 9).

As indicated in FIG. 8, shield 32 is constructed from a UV radiation-opaque material, such as polycarbonate, polystyrene and like materials that will effectively shield the surrounding tissue so that only the target areas of the patient will receive the germicidal UV energy.

It is to be understood that the unique design of the probe cover as shown in the drawings not only provides protection for the UV light probe 20 and a method of preventing probe contamination, the probe also sets the proper distance from the light source to the tissue being exposed to the UV radiation. This factor is especially important because the energy transmitted to the tissue decreases exponentially with the distance between the light source and the tissue surface. In this regard, it should be appreciated that in carrying out the method of the invention for treating bacterial, viral, fungal, and parasitic infections in the patient's tissue, pre-setting the distance between light source and tissue in this manner is quite different than simply holding a UV light over the tissue as this approach does not stretch the tissue as does the UV light probe 20. In accordance with one form of the method of the invention, the tissue is uniformly stretched so that the tissue is uniformly exposed to the light energy which is critical to the successful germicidal use of UV light as it is a ‘line of sight’ process. Creases or folds can hide bacteria from exposure to the light energy, preventing them from receiving a lethal dose of irradiation.

Additionally, the probe covers of the apparatus are designed so that they slightly dilate the lumen of the orifice into which they are inserted, such as the vagina and rectum. In accordance with the method of the invention, this dilation sets the distance from light source to tissue surface very accurately as the dilated tissue conforms to the probe cover, giving uniform coverage of the UV light energy to the tissue to be treated.

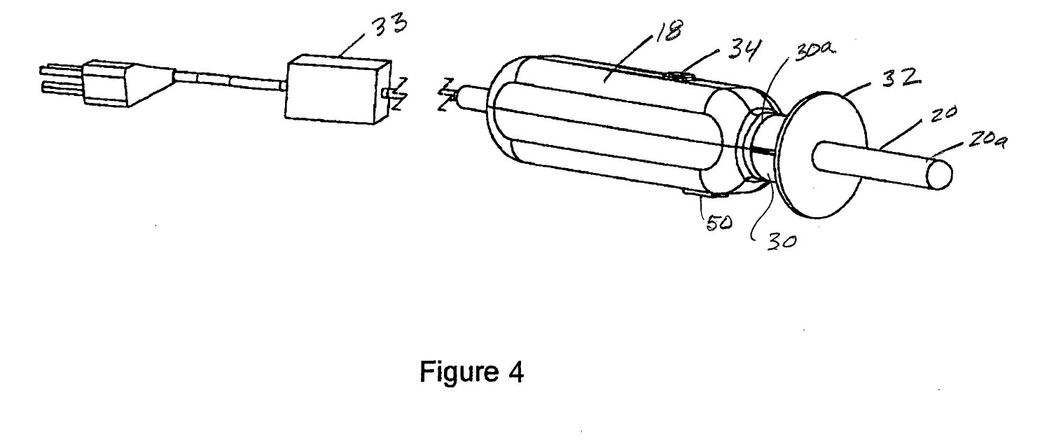

Carried by housing 16 is switching means for controllably energizing the source of UV radiation 22, which here comprises a conventional, readily commercially available UV-generating lamp. In the present form of the invention this switching means, which comprises a part of the electrical circuitry of the invention (see FIG. 5), is provided in the form of a conventional, readily commercially available, one-touch activator switch 34. Switch 34 is mounted on the exterior wall 16c of the housing 16 (FIG. 1) and, as shown in FIGS. 4 and 5, is operably interconnected in a conventional manner with a source of electrical power 36 and with the UV radiation source 22 via a conventional transformer 33, a conventional relay 35, the timer 24 and the novel disabling means of the invention, the character of which will presently be described. Timer 24, which ensures that the tissue is irradiated for a predetermined period of time, is here provided in the form of a conventional, readily commercially available, adjustable timer that can be pre-set to control the length of time that the UV radiation source remains energized after the circuit is closed. Radiation source 22, timer 24, switch 34, relay 35 and transformer 36 are interconnected within the electrical circuitry in a manner well understood by those skilled in the art.

An important feature of the present invention is the provision of a novel combination cover-ejecting means and safety-disabling means. The cover-ejecting means permits the operator to safely eject the cover after it becomes contaminated without touching the cover. The novel safety-disabling means here functions to prevent energizing the UV radiation source by the switching means of the invention unless the probe cover 26 is correctly in position over the probe 20. In the present form of the invention this novel combination cover-ejecting means and safety-disabling means comprises an assembly generally designated by the numeral 40 that is carried by housing 16 in the manner best seen in FIGS. 2 and 5. In this embodiment of the invention, assembly 40 comprises an ejector member 42 that is movable by portion 30 of the cover 20 from a first extended position shown in FIG. 2 to a second retracted position shown in FIG. 5.

With the ejector member having been moved by the rim portion 30a of the cover (see FIGS. 3, 4 and 5) into the second retracted position shown in FIG. 5, the electrical circuit between points 44 and 46 is completed so that the source of radiation can be energized by the operation of switch 34. Once energized, the UV radiation source will remain in its energized state for the period of time set on the timer 24. At the expiration of this time, the UV energy source will be automatically de-energized and the probe cover can be safely ejected from the hub portion 18b of the housing.

It is to be noted that as the probe cover is ejected from the hub portion 18b of the housing, the ejector member is urged by the biasing means, or spring 48, into the extended position shown in FIG. 2, thereby breaking the electrical circuit between points 44 and 46 so that the source of radiation 22 cannot be re-energized by the operation of switch 34.

On the other hand, as can be seen by referring to FIGS. 2 and 5, movement of the ejector member toward its retracted position by positioning the cover 26 over the probe 20 in the manner shown in FIGS. 4 and 5, will once again complete the electrical circuit between points 44 and 46.

Movement of the ejector member into the retracted position shown in FIG. 5 will also move a finger-engaging ejector slide assembly 49, which is slidably connected to housing 16, rearwardly of the housing against the urging of spring 48. Finger-engaging ejector slide assembly 49, which comprises a part of the cover-ejecting means of the invention, includes a finger-engaging ejector slide 52 and an integrally formed ejector frame 54. Portion 54a of ejector frame 54 is in operable engagement with spring 48, while portion 54b of the frame is in operable engagement with ejector member 42. With this construction, after the irradiation treatment has been completed, the contaminated cover can be readily ejected from the hub portion 18b by merely sliding the finger-engaging ejector slide 52 forwardly of the housing. In this regard, as the finger-engaging ejector slide 52 moves forwardly, the ejector member 42 will be caused to move toward its extended position and, in so doing, will safely eject the contaminated cover from the hub portion 18b of the housing. In this way, the operator can safely and conveniently eject the contaminated cover into an appropriate disposal container without touching it.

In using the device of the invention to carry out the method of the invention, the user first positions a clean probe cover 26 over the probe in the manner shown in FIGS. 4 and 5. This done, timer 24 is set and the tip portion of the cover is inserted into the body orifice, such as the patient's nostril, in the manner illustrated in FIG. 14 for a timed application to the nose of germicidal UV energy.

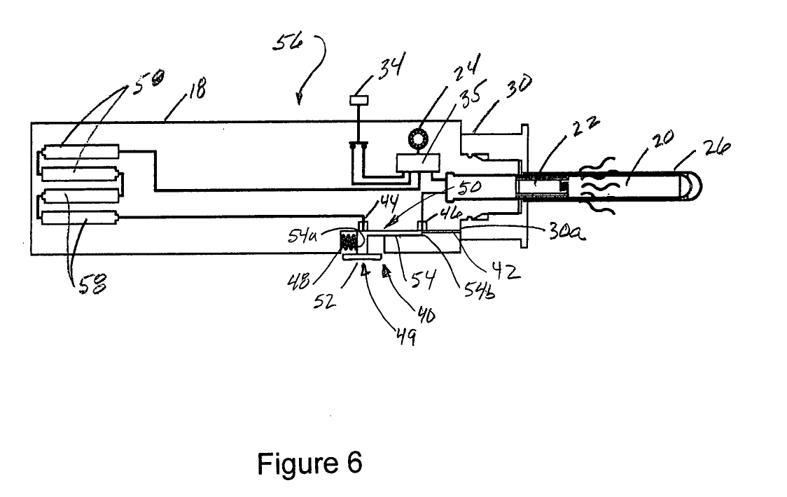

Turning to FIG. 6, an alternate form of the apparatus of the invention is there shown and generally designated as 56. This alternate form of the invention is identical in construction and operation to the embodiment of FIGS. 1 through 5, save for the use of a different source of electrical power. As depicted in FIG. 6, where like numerals have been used to identify like components, the source of electrical power here comprises a plurality of conventional, appropriately interconnected batteries 58. Batteries 58 are interconnected with the electrical circuitry in the manner shown in FIG. 6 and provide power to the UV energy source 22.

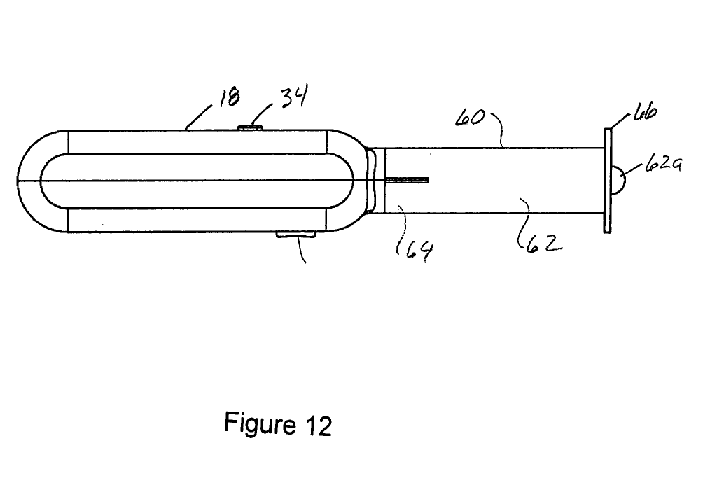

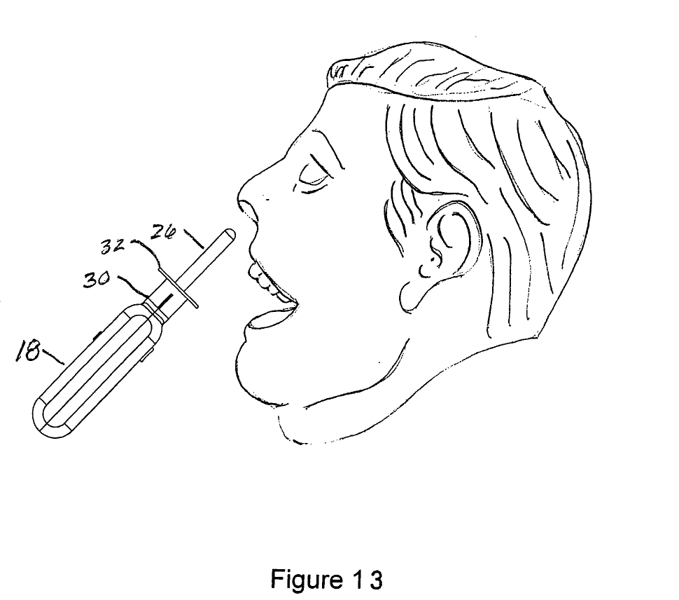

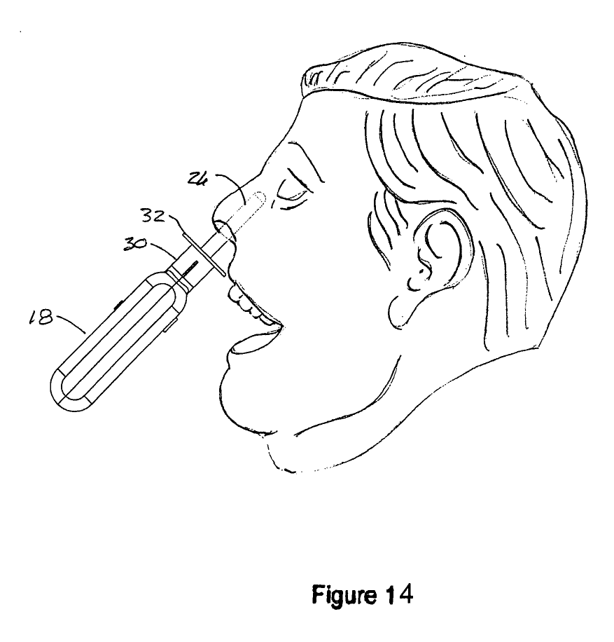

For treatment of skin lesions, the user replaces the probe cover 26 of the apparatus with an appropriately sized and shaped skin contact cover 60 of the character shown in FIGS. 11 and 12. Substantially UV radiation-transparent probe cover 60, which is similar in construction and operation to cover 26, here comprises a generally tubular-shaped forward portion 62 that covers the forward portion of the probe and a generally tubular-shaped, slotted connector portion 64 that is telescopically receivable over the forward hub portion 18b hub of housing portion 18. Forward portion 62, which includes a tip cover portion 62a, can be constructed from polypropylene, certain varieties of Teflon that are transparent to UV light in the UV-C range (200-280 nm) and other amorphous fluropolymers. Disposed proximate tip portion 62a is an enlarged diameter radiation shield 66 that functions to shield the user's hand from radiation (see FIGS. 11 and 12). As before, shield 66 is constructed from a UV radiation-opaque material, such as polycarbonate, polyethylene and like materials that will effectively shield the surround tissue so that only the target areas of the patient will receive the germicidal UV energy.

The alternate form of the invention shown in FIG. 12 is identical in construction and operation to the embodiment of FIGS. 1 through 5, save for the use of the differently configured probe cover 60.

In certain instances, germicidal UV energy may be selectively applied both to various body orifices, such as the nostril, as well as to the skin of the patient using the probe cover 70 illustrated in FIG. 10 of the drawings. Probe cover 70, which is also similar in construction and operation to cover 26, here comprises a generally tubular-shaped forward portion 72 that covers the forward portion of the probe and a generally tubular-shaped, slotted connector portion 74 that is telescopically receivable over the forward hub portion 18b hub of housing portion 18. Forward portion 72, can be constructed from polypropylene, certain varieties of Teflon that are transparent to UV light in the UV-C range (200-280 nm) and other amorphous fluropolymers. Disposed intermediate forward portion 72 and slotted connector portion 74 is an enlarged diameter radiation shield 76 that functions to shield the user's hand from radiation. As before, shield 76 is constructed from a UV radiation-opaque material such as polycarbonate, polyethylene and like materials that will effectively shield the surrounding tissue so that only the target areas of the patient will receive the germicidal UV energy.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

We claim:

1. An irradiation device for treating bacterial, viral, fungal and parasitic infections found on the skin and in the anatomical orifices of a patient comprising:
(a) a housing including a hollow body;
(b) a probe connected to said housing and extending therefrom;
(c) a source of UV radiation operably associated with said probe for emitting UV radiation in a direction towards said forward irradiating portion of said probe; and
(d) a substantially UV radiation-transparent probe cover removably connected to said housing for covering said probe.

2. The device as defined in claim 1 which said housing includes a forward hub portion and in which said probe cover includes a connector portion telescopically receivable over said hub portion of said housing.

3. The device as defined in claim 1 in which said source of UV radiation comprises a UV-generating lamp disposed within said probe.

4. The device as defined in claim 1 in which said probe cover includes a substantially UV-C-opaque safety shield.

5. The device as defined in claim 1, further including an ejector means carried by said housing for ejecting said probe cover from said housing.

6. The device as defined in claim 1, further including timer means carried by said housing for controlling the length of time that a said source of UV radiation is energized.

7. The device as defined in claim 1, further including switching means carried by said housing for energizing said source of UV radiation.

8. The device as defined in claim 6, further including disabling means carried by said housing for preventing energizing said source of UV radiation by said switching means unless said probe cover is covering said probe.

9. An irradiation device for treating bacterial colonies on the skin of the patient and in body orifices of the patient comprising:
(a) a housing including a hollow body;
(b) an elongated probe connected to said housing and extending therefrom, said elongated probe having a forward irradiating portion for insertion into the body orifice and a rearward portion;
(c) a UV-generating lamp disposed within said probe for emitting UV radiation in a direction towards said irradiating portion of said elongated probe;
(d) timer means carried by said housing for controlling the length of time that said UV-generating lamp is energized;
(e) switching means carried by said housing for energizing said UV-generating lamp; and
(f) a substantially UV radiation-transparent probe cover removably connected to said housing for covering said elongated probe.

10. The device as defined in claim 9 which said housing includes a forward hub portion and in which said probe cover includes a connector portion telescopically receivable over said hub portion of said housing.

11. The device as defined in claim 10, further including a combination cover-ejecting means and safety-disabling means carried by said housing for ejecting said cover from said hub portion of said housing and for preventing said UV-generating lamp from being energized by said switching means of the invention unless said probe cover is in position over said hub portion of said housing.

12. The device as defined in claim 11 in which said combination cover-ejecting means and safety-disabling means comprises an ejector member movable by said hub portion of said housing from a first extended position to a second retracted position.

13. The device as defined in claim 12 in which said combination cover-ejecting means and safety-disabling means further comprises biasing means for yieldably resisting movement of said ejector member toward said second retracted position.

14. The device as defined in claim 13 in which said combination cover-ejecting means and safety-disabling means further comprises a finger-engaging ejector slide carried by said housing for moving said ejector member toward said first extended position.

15. An irradiation device for treating bacterial colonies in the nose and on the skin of a patient comprising:
(a) a housing including a hollow body portion and a forward hub portion;
(b) an elongated probe connected to said housing and extending therefrom, said elongated probe having a forward irradiating portion and rearward portion;
(c) a UV-generating lamp disposed within said probe for emitting UV radiation in a direction towards said irradiating portion of said elongated probe;
(d) timer means carried by said housing for controlling the length of time that said UV-generating lamp is energized;
(e) switching means carried by said housing for energizing said UV-generating lamp; and
(f) a substantially UV radiation-transparent probe cover removably connected to said housing for covering said elongated probe, said probe cover including a connector portion telescopically receivable over said hub portion of said housing; and
(i) a combination cover-ejecting means and safety-disabling means carried by said housing for ejecting said cover from said hub portion of said housing and for preventing said UV-generating lamp from being energized by said switching means of the invention unless said probe cover is in position over said hub portion of said housing.

16. The device as defined in claim 15 in which said probe cover includes a substantially UV-C-opaque safety shield.

17. The device as defined in claim 15 in which said hollow body portion of said housing is constructed from a substantially UV-C-opaque material.

18. The device as defined in claim 15 in which said combination cover-ejecting means and safety-disabling means comprises an ejector member movable by said hub portion of said housing from a first extended position to a second retracted position.

19. The device as defined in claim 18 in which said combination cover-ejecting means and safety-disabling means further comprises biasing means for yieldably resisting movement of said ejector member toward said second retracted position.

20. The device as defined in claim 19 in which said combination cover-ejecting means and safety-disabling means further comprises a finger-engaging ejector slide carried by said housing for moving said ejector member toward said first extended position.

21. A method for treating bacterial, viral, fungal and parasitic infections in tissue located proximate a body cavity of a patient using an elongated probe that is operably associated with a source of UV radiation, comprising the steps of inserting the probe into the body cavity to controllably stretch the cavity and to accurately position the source of UV radiation relative to the tissue to ensure that the radiation reaches all tissue surfaces and that the tissue is uniformly exposed to the radiation.

22. The method defined in claim 21 in which the tissue is irradiated for a predetermined period of time.
